(19) United States
(12) Patent Application Publication
(10) Pub. No.: US 2025/0255748 A1
(43) Pub. Date: Aug. 14, 2025

(54) THERMAL TREATMENT DEVICE AND METHOD OF USE

(71) Applicant: Lilliam Civantos, Hinsdale, IL (US)

(72) Inventor: Lilliam Civantos, Hinsdale, IL (US)

(21) Appl. No.: 18/441,662

(22) Filed: Feb. 14, 2024

(51) Int. Cl. A61F 7/02; A61F 7/00; A61F 7/10; A61F 13/08

(52) U.S. Cl. CPC A61F 7/02; A61F 13/085; A61F 2007/0039; A61F 2007/0219; A61F 2007/0231; A61F 2007/0238; A61F 2007/108

(57) ABSTRACT

A thermal treatment device for thermal treatment of human leg is disclosed. The thermal treatment device comprises a sleeve for covering a back of the human leg, and at least three pockets adjacent to a glute, a hamstring, and a calf of the leg. The sleeve is configured to be maintained on the leg. The pockets are adaptable to receive a thermal source. The sleeve covers the back of the leg from a calf to the glute and a front of the leg from a shin to a groin. The sleeve is a split sleeve having a first edge and a second edge. The sleeve is configured to be wrapped around the leg to cover the back of the leg.

### FIELD

The field is related to a thermal treatment device. The field may particularly relate to a thermal treatment device and a method of thermally treating a human leg.

### BACKGROUND

Thermal therapy conventionally involves the application of heat or cold to body tissue in an effort to heal and rehabilitate injuries such as bruises, sprains, or other trauma to bone, muscle, ligaments, tendons, and other tissue and to treat degenerative conditions and inflammatory diseases and disorders. In a cold application, a cold medium is applied to an affected bodily area to diminish swelling and inflammation so to reduce pain and promote healing of injured tissue. In a heat application, a hot medium is applied to an affected bodily area to loosen extremity tissue and joint tissue, such as muscles, ligaments, and tendons.

Application of heat promotes repair and healing by increasing the temperature of the affected area, thereby increasing the flow of blood and oxygen to the tissue and increasing respiration. In this manner, the application of heat serves to increase the range of motion and improve the flexibility in the patient's extremity, thus improving the functionality, comfort, and performance of the injured or infirm targeted area.

Application of cold diminishes inflammation of tissue. Chilling tissue causes vasoconstriction in which blood vessels contract and drain blood from chilled areas. After removing the chilling device from the vicinity of the tissue, fresh blood flows into the previously contracted vessels, invigorating the muscles with oxygen and in turn, reducing inflammation. This increase in blood flow also makes its way deep into the muscle tissue, helping flush out any built-up lactic acid. The combined effect of supplying oxygen to muscles while flushing out lactic acid facilitates recovery of muscles, tendons, bones, nerves, and other tissue.

A chilling method of thermal therapy which many people have resorted to is an ice bath. Ice baths can carry disadvantages. For example, the ice bath causes intense discomfort and severe pain owing to the frigid ice and water. Another disadvantage is that an ice bath indiscriminately chills the entire submerged portions of the body. Therefore, not only are the affected muscle groups exposed to the cold temperatures, but all neighboring parts of the anatomy must endure the intense cold of an ice bath, even if no benefit is attainable for those neighboring parts of the body. Additionally, ice baths are most effective when taken within a short period of time after an intense activity. However, ice baths are not typically portable and are often located remote from or inaccessible after intense activity.

Other conventional thermal therapies do not facilitate targeting a related group of muscles, ligaments, tendons, and tissues simultaneously. Instead, such other conventional thermal therapies typically involve wrapping a band around the heat source and a part of the body to attempt to maintain the heat source on the targeted bodily area. Applying a wrap to secure a heat source on a targeted bodily area may be difficult, and the heat source frequently shifts away from the targeted area. Consequently, these conventional therapies may only attempt to target only one part of the related muscle group and often imprecisely.

Sciatica is a symptom that refers to pain, weakness, numbness, or tingling in the legs, due to damage or pressure to the sciatic nerve. Sciatic nerves originate from the spinal cord at the lower spine and innervate various parts of the legs. The condition also gets worse with activities that increase the spinal load in addition to other causes. Spinal decompression is one of the usual methods used to treat sciatica. However, these usual methods are unable to off-load pressure from the vertebral column effectively.

Thus, there exists a need for an improved thermal treatment device and a method for targeting a group of muscles, ligaments, tendons, and other tissues simultaneously with a thermal medium.

### SUMMARY

A thermal treatment device and a method for thermally treating a human leg are disclosed. The thermal treatment device may comprise a sleeve which is configured to be wrapped around the leg to cover the back of the leg. The thermal treatment device has a first pocket located adjacent to a glute, a second pocket adjacent to a hamstring, and a third pocket adjacent to a calf of the leg. The pockets are adapted to receive a thermal source. The thermal treatment device may have a plurality of bands for wrapping the sleeve around the leg. The thermal treatment device may be specifically used in a method to apply sequential cooling and heating to the back of the leg to treat a sciatica condition.

### BRIEF DESCRIPTION OF THE DRAWINGS

FIG. 1 is a front perspective view of the thermal treatment device maintained on a human leg in accordance with an embodiment of the present disclosure.

FIG. 2 is a rear perspective view of the thermal treatment device wherein the thermal treatment device is open and ready to be wrapped around the leg in accordance with another embodiment of the present disclosure.

FIG. 3 is a front perspective view of the thermal treatment device wherein the thermal treatment device is open and ready to be wrapped around the leg in accordance with another embodiment of the present disclosure.

FIG. 4 is a side perspective view of the thermal treatment device maintained on the human leg in accordance with another embodiment of the present disclosure.

FIG. 5 is a rear perspective view of the thermal treatment device maintained on the human leg in accordance with yet another embodiment of the present disclosure.

FIG. 6 is another side perspective view of the thermal treatment device maintained on the human leg in accordance with another embodiment of the present disclosure.

### DETAILED DESCRIPTION

In accordance with the present disclosure, a thermal treatment device is disclosed for providing a thermal treatment to a human leg. The thermal treatment device of the present disclosure is adapted for a human body, and may be dimensioned to accommodate different types, shapes and sizes of the human body sizes and its contours.

The thermal treatment device of the present disclosure can be wrapped around the leg to provide a heating or a cooling effect or both to the leg. The thermal treatment device of the present disclosure provides a thermal effect to the leg from the lower back to the ankle of the leg. The thermal treatment device of the present disclosure can be used to thermally treat a group of muscles, ligaments, tendons, and other tissues of the lower back and of the leg simultaneously with a thermal medium that can be placed in the thermal device and maintained in contact with the skin in the vicinity of the target area.

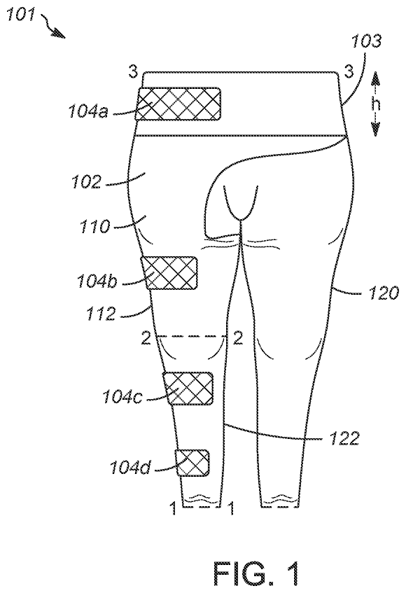

Referring to FIG. 1, a front view of a thermal treatment device 101 configured in accordance with an embodiment of the present disclosure is disclosed. In an aspect, the thermal treatment device 101 comprises a sleeve 102 for covering a human leg 110. The thermal treatment device 101 also comprises a plurality of pockets to receive a thermal source located at predetermined positions on the sleeve 102 for providing thermal treatment to the leg 110. In accordance with an exemplary embodiment of the present disclosure, the thermal treatment device 101 may be a split sleeve 102. In accordance with another exemplary embodiment of the present disclosure, the thermal treatment device 101 may be a compression split sleeve 102. The thermal treatment device 101 may be a form fitting split sleeve 102 that can extend from the waist to just above the ankle of a human leg 110. In other embodiments, the thermal treatment device 101 may be a form fitting sleeve 102 without a split.

In another aspect of the present disclosure, the sleeve 102 may be a split brace for covering a human leg 110. The sleeve 102 may comprise a suitable rigid material or include one or more plastic slats extending longitudinally along the sleeve for providing support to immobilize the leg 110. The slats may be removably associated with the split sleeve 102 such as by being provided in pockets or removably fastened to the leg 110.

The sleeve 102 is preferably maintained around the leg 110. In an embodiment as shown in FIG. 1, the sleeve 102 is maintained around a right leg 110. The sleeve 102 may cover the back of the leg 110 from a calf to the glute. The sleeve 102 may cover a front of the leg from a shin to a groin. The different heights of the leg 110 are shown with the dotted lines in the Figures. The ankle is shown with a dotted line 1-1, the knee joint is shown with a dotted line 2-2 and the waist is shown with a line 3-3 in FIG. 1. The thermal treatment device 101 of the present disclosure is provided with a plurality of bands, a first band 104a, a second band 104b, a third band 104c, and a fourth band 104d for securing the sleeve 102 around the leg, so that the thermal treatment device 101 is maintained on the human leg 110.

In an embodiment, the sleeve 102 comprises a belt 103 that may extend across a human waist. The belt 103 may be made of the same material as the split sleeve 102. The belt 103 may be a continuous part of the sleeve 102 and is integrated with the sleeve 102. The belt 103 may be of a suitable or an adjustable height “h” near the waistline 3-3 for securing the belt to the waist.

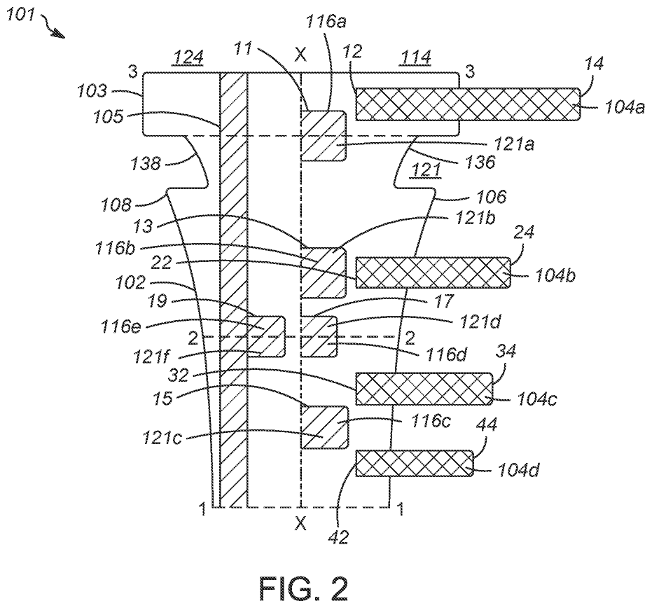

A rear plan view of thermal treatment device depicting the outer view of the split sleeve 102 is shown in FIG. 2. As shown in FIG. 2, the split sleeve 102 is open and is adaptable to be wrapped around the leg. A first edge 106 and a second edge 108 on opposite sides of the sleeve 102 are shown in FIG. 2. An imaginary axis X-X demarks the middle line of the sleeve 102 between the first edge 106 and the second edge 108. In an exemplary embodiment, four bands, the first band 104a, the second band 104b, the third band 104c, and the fourth band 104d are shown. The split sleeve 102 has a rear or outside surface 121 which has the first side 114 and the second side 124 demarked by the imaginary axis X-X. In an embodiment, the first band 104a extends at the same elevation as the belt across the waistline 3-3 to secure the sleeve 102 to the waist. The sleeve 102 has a first recess 136 on the first side 114 below the first band 104a and/or the belt 103 and above the second band 104b. The sleeve 102 has a second recess 138 on the second side 124 below the belt 103 and above the second band 104b. The first recess 136 and the second recess 138 are provided in the sleeve 102 on the thigh area to enable the person wearing the sleeve 102 to move or sit comfortably. The first recess 136 and the second recess 138 expose the vicinity of the groin when worn on the leg 110.

The first band 104a has a first end 12 and a second end 14, the second band 104b has a first end 22 and a second end 24, the third band 104c has a first end 32 and a second end 34, and the fourth band 104d has a first end 42 and a second end 44. The first end 12 of the first band 104a, the first end 22 of the second band 104b, the first end 32 of the third band 104c, and the first end 42 of the fourth band 104d are secured to the outside surface 121 of the first side 114 of the split sleeve 102. The second end 14 of the first band 104a, the second end 24 of the second band 104b, the second end 34 of the third band 104c, and the second end 44 of the fourth band 104d are adaptable to be removably secured to the outside surface 121 of the second side 124 of the split sleeve 102. A patch or strip 105 of mating fasteners is affixed to the second side 124 of the split sleeve 102 for matingly engaging with fasteners on the second end of the respective band. Although, a single strip 105 of mating fasteners is shown, separate dedicated patches of mating fasteners may be fastened to the outside surface 121 of the second side for each of the plurality of bands the first band 104a, the second band 104b, the third band 104c, and the fourth band 104d, respectively.

At least three pockets, a first pocket 116a, a second pocket 116b, and a third pocket 116c are also shown in FIG. 2. The first pocket 116a, the second pocket 116b, and the third pocket 116c are located on the outside surface 121 of the sleeve for receiving a suitable thermal source. In an embodiment, the first pocket 116a, the second pocket 116b, and the third pocket 116c are located on the first side 114 of the sleeve 102 as shown in FIG. 2. The rear side of the first pocket 116a, the second pocket 116b, and the third pocket 116c are depicted in FIG. 2. The first pocket 116a has a rear side 121a, the second pocket 116b has a rear side 121b, and the third pocket 116c has a rear side 121c. For accessibility, the first pocket 116a is open on the top side 11, the second pocket 116b is open on the top side 13, and the third pocket 116c is open on the top side 15 of the respective pocket. Alternatively, the first pocket 116a, the second pocket 116b, and the third pocket 116c may be open from any side as per suitability. The first pocket 116a, the second pocket 116b, and the third pocket 116c may be configured to include a zipper or any suitable closure for closing the pockets after placing a thermal source inside the pockets to secure the thermal source inside the pocket. The first pocket 116a, the second pocket 116b, and the third pocket 116c may be configured to include a fastener on the inner side of the pocket. The fastener may be selected from strips comprising a plurality of hooks that may matingly engage a plurality of loops such as Velcro or may comprise other disengageable fastening devices such as snaps, zippers, or buttons. However, the first pocket 116a, the second pocket 116b, and the third pocket 116c may be kept open without a closure for ease of accessibility.

In an embodiment, a fourth pocket 116d and a fifth pocket 116e are also shown in FIG. 2. The rear side of the fourth pocket 116d and the fifth pocket 116e is also depicted in FIG. 2. The fourth pocket 116d has a rear side 121d, and the fifth pocket 116e has a rear side 121f. For accessibility, the fourth pocket 116d is open on the top side 17 and the fifth pocket 116e is open on the top side 19. Alternatively, the fourth pocket 116d and the fifth pocket 116e may be open on any side as per suitability. The fourth pocket 116d and the fifth pocket 116e may be configured to include a zipper or any suitable closure for closing the pockets after placing the thermal source inside the pockets to secure the thermal source when the thermal source is inside the pocket. However, the fourth pocket 116d and the fifth pocket 116e may be kept open for ease of accessibility. In an aspect, the fourth pocket 116d and the fifth pocket 116e are optional.

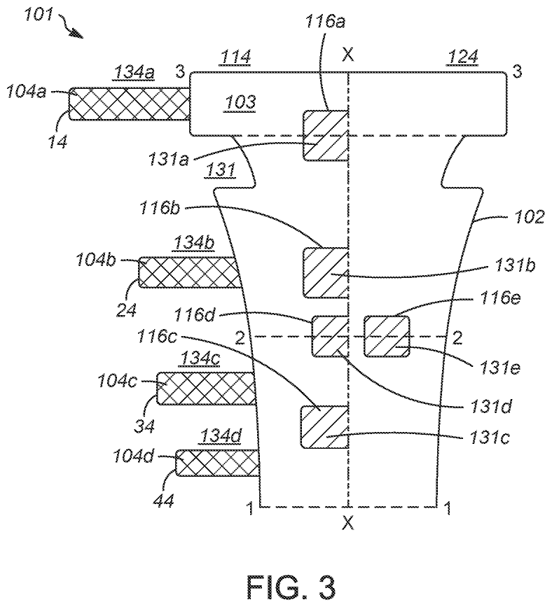

A front plan view of the open thermal treatment device depicting the inside surface 131 of the split sleeve 102 is shown in FIG. 3. As shown in FIG. 3, the split sleeve 102 is open and is ready to be wrapped around the leg. The split sleeve 102 has a front or inside surface 131 which has the first side 114 and the second side 124 demarked by the imaginary axis X-X as shown in FIG. 3. The first pocket 116a, the second pocket 116b, and the third pocket 116c are shown in FIG. 3. The inner side of the first pocket 116a, the second pocket 116b, and the third pocket 116c is depicted in FIG. 3. The first pocket 116a has an inner side 131a, the second pocket 116b has an inner side 131b, and the third pocket 116c has an inner side 131c. The first pocket 116a, the second pocket 116b, and the third pocket 116c are accessible from the outside surface 121 of the sleeve 102 as described previously. The fourth pocket 116d and the fifth pocket 116e are also shown. The fourth pocket 116d has an inner side 131d, and the fifth pocket 116e has an inner side 131e. FIG. 3 also shows an open rear view of the belt 103 of the sleeve 102. The plurality of bands 104a, 104b, 104c and 104d may include one or more fasteners on an inside surface 134a of the second end 14 of the first band 104a, an inside surface 134b of the second end 24 of the second band 104b, an inside surface 134c of the second end 34 of the third band 104c, and an inside surface 134d of the second end 44 of the fourth band 104d as shown in FIG. 3. The fasteners may be selected from strips comprising a plurality of hooks that may matingly engage a plurality of loops such as Velcro or may comprise other disengageable fastening devices such as snaps, zippers, or buttons. As explained, an appropriate mating fastener may be located on the strip 105 on the second side 124 of the outside surface 131 of the split sleeve 102, such that the second end 14 of the first band 104a, the second end 24 of the second band 104b, the second end 34 of the third band 104c, and the second end 44 of the fourth band 104d can be mated with the mating fastener to hold the bands around the leg and the sleeve closed about the split. Although, in the FIGURES, the thermal treatment device 101 is provided with four bands the first band 104a, the second band 104b, the third band 104c, and the fourth band 104d on the sleeve 102, there may be more or less than four bands on the sleeve 102 to maintain the thermal treatment device 101 on the leg. The first band 104a, the second band 104b, the third band 104c, and the fourth band 104d secure the sleeve 102 on the leg and maintain the sleeve 102 in a wrapped condition.

In an alternate embodiment, the first band 104a, the second band 104b, the third band 104c, and the fourth band 104d may be placed alternatively on the first side 114 and the second side 124 of the outside surface 121. In the alternate embodiment, for example, the first end 12 of the first band 104a, and the first end 32 of the third band 104c may be secured to the first side 114, and the first end 22 of the second band 104b, and the first end 42 of the fourth band 104d may be secured to the second side 124 of the outside surface 121. The mating fasteners for the second end 14 of the first band 104a, and the second end 34 of the third band 104c can be fastened on the second side 124, and the mating fasteners for the second end 24 of the second band 104b, and the second end 44 of the fourth band 104d can be fastened to the first side 114. In another alternate embodiment, the first end 12 of the first band 104a, and the first end 32 of the third band 104c may be fastened to the second side 124, and the first end 22 of the second band 104b, and the first end 42 of the fourth band 104d may be fastened to the first side 114 of the outside surface 121. The mating fasteners for the second end 14 of the first band 104a, and the second end 32 of the third band 104c can be fastened on the first side 114, and the mating fasteners for the second end 24 of the second band 104b, and the second end 44 of the fourth band 104d can be fastened to the second side 124.

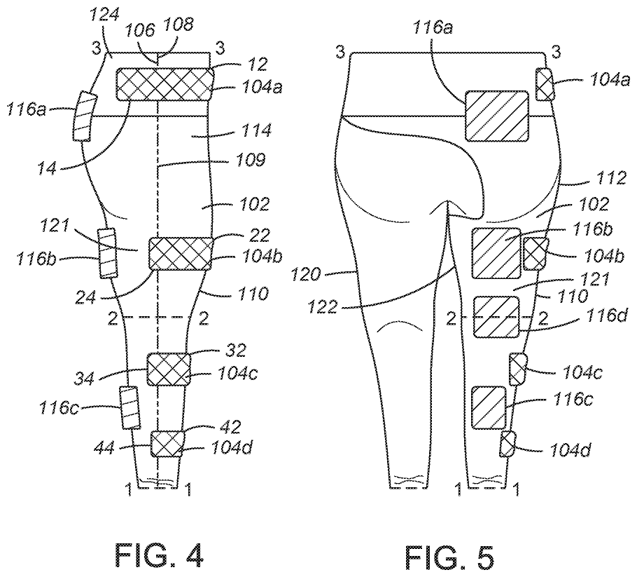

FIG. 4 is a side view of the split sleeve 102 maintained on the leg 112. In an exemplary embodiment, the split sleeve 102 has the first edge 106 on the first side 114 and the second edge 108 on the second side 124 of the split sleeve 102. When wrapped around the leg 110, the first edge 106 and the second edge 108 define a split 109. The split sleeve 102 is configured to be wrapped around the leg 110 in one embodiment to cover both the back and the front of the leg. In another embodiment, the sleeve 102 may be wrapped around the leg to partially cover the leg. When the sleeve 102 is wrapped around the leg 110, the first edge 106 and the second edge 108 may lie opposite to each other to define a split 109 on the outer side 112 (FIG. 1) of the leg 110. Alternatively, the split sleeve 102 may be configured such that when wrapped around the leg 110, the first edge 106 and the second edge 108 of the split sleeve 102 may lie opposite to each other to define a split on the inner side 122 (FIG. 1) of the leg 110. In another embodiment, the first edge 106 and the second edge 108 may be spaced apart from each other to define a wider split 109. A side perspective view of the first band 104a, the second band 104b, the third band 104c, and the fourth band 104d is also shown. FIG. 4 also depicts a side perspective view of the first pocket 116a, the second pocket 116b, and the third pocket 116c.

When the split sleeve 102 is wrapped around the leg, the sleeve encases the leg such that the first edge 106 of the split sleeve is opposed to the second edge 108 of the split sleeve 102. The first band 104a, the second band 104b, the third band 104c, and the fourth band 104d are provided on the outside surface 121 of the split sleeve 102 to traverse the split 109 while wrapping around the sleeve 102 to maintain the sleeve on the leg.

Referring to FIG. 4, the thermal treatment device 101 is shown wrapped around a right leg 110. The plurality of bands, the first band 104a, the second band 104b, the third band 104c, and the fourth band 104d traverse from the first side 114 across the split 109 to the second side 124. The first band 104a has the first end 12 fastened to the outer surface 121 of the first side 114 of the sleeve 102, wraps around the leg 110 across the split 109 and fasteners on an inside surface 134a (FIG. 3) of the second end 14 engage a strip 105 (FIG. 2) of mating fasteners on the outer surface 121 of the second side 124. The second band 104b has the first end 22 fastened to the outer surface 121 of the first side 114 of the sleeve 102, wraps around the leg 110 across the split 109 and fasteners on an inside surface 134b (FIG. 3) of the second end 24 engage a strip 105 (FIG. 2) of mating fasteners on the outer surface 121 of the second side 124. The third band 104c has the first end 32 fastened to the outer surface 121 of the first side 114 of the sleeve 102, wraps around the leg 110 across the split 109 and fasteners on an inside surface 134c (FIG. 3) of the second end 34 engage a strip 105 (FIG. 2) of mating fasteners on the outer surface 121 of the second side 124. The fourth band 104d has the first end 42 fastened to the outer surface 121 of the first side 114 of the sleeve 102, wraps around the leg 110 across the split 109 and fasteners on an inside surface 134d (FIG. 3) of the second end 44 engage a strip 105 (FIG. 2) of mating fasteners on the outer surface 121 of the second side 124. The thermal treatment device 101 can be equally maintained on a left leg 120 (FIG. 1) with the plurality of bands, the first band 104a, the second band 104b, the third band 104c, and the fourth band 104d, on the split sleeve 102.

Referring back to FIG. 3 in conjunction with FIG. 4, when the sleeve 102 is secured on the leg, the inner side 131a of the first pocket 116a, the inner side 131b of the second pocket 116b, and the inner side 131c of the third pocket 116c are in direct contact with the leg. Particularly, the inner side 131a of the first pocket 116a is in direct contact with the glute muscle area of the leg, the inner side 131b of the second pocket 116b is in direct contact with the hamstring muscle area of the leg, and the inner side 131c of the third pocket 116c is in direct contact with the calf muscle area of the leg.

In accordance with the present disclosure, the sleeve 102 may be made of tight flexible fabric, such as polyester, elastane, or spandex or it may be made of a looser fabric. When maintained on the leg 110, the sleeve 102 covers the back of the leg from a calf to the glute and the front of the leg from a shin to a groin as depicted in FIG. 1.

FIG. 5 is a rear view of the sleeve 102 maintained on the right leg 110. In an exemplary embodiment, the three pockets, the first pocket 116a, adjacent to a glute, the second pocket 116b adjacent to a hamstring, and the third pocket 116c adjacent to a calf of the leg 110 when the sleeve is worn on the leg are shown in FIG. 5. The first pocket 116a, the second pocket 116b, and the third pocket 116c are adaptable to receive a thermal source for providing a thermal treatment to the leg 110. The sleeve 102 may comprise more or less than three pockets 116a, 116b, and 116c for receiving the thermal sources. In an aspect, the split sleeve 102 may comprise a fourth pocket 116d located behind the kneecap to provide thermal treatment to the underside of the knee. In an exemplary embodiment, the fourth pocket 116d located behind the kneecap has a smaller size than the first pocket 116a, the second pocket 116b, and the third pocket 116c.

In accordance with an aspect of the present disclosure, the first pocket 116a, the second pocket 116b, and the third pocket 116c are configured on the outside surface 121 of the sleeve 102. The pockets 116 are adaptable to receive a thermal source to provide heating or cooling to the leg 110, particularly, the first pocket 116a is adaptable to receive a thermal source to provide heating or cooling to the glute, the second pocket 116b is adaptable to receive a thermal source to provide heating or cooling to the hamstring, and the third pocket 116c is adaptable to receive a thermal source to provide heating or cooling to the calf at the back of the leg 110. A heated or a cooled thermal source can be placed inside the first pocket 116a, the second pocket 116b, and the third pocket 116c for providing the thermal treatment to the leg 110. In an exemplary embodiment, the first pocket 116a, the second pocket 116b, and the third pocket 116c are accessible on the outside surface 121 of the split sleeve 102. The split sleeve 102 can be wrapped on the leg 110 and secured to the leg with the first band 104a, the second band 104b, the third band 104c, and the fourth band 104d such that the inner side 131a of the first pocket 116a, the inner side 131b of the second pocket 116b, and the inner side 131c of the third pocket 116c on the inside surface 121 of the sleeve 102 are in contact with the leg 110. Thereafter, a heated or a cooled thermal source can be placed into the first pocket 116a, the second pocket 116b, and the third pocket 116c which are accessible on the outside surface 121 of the sleeve 102. When the sleeve 102 is wrapped around the leg 110, the inside surface 131 of the sleeve 102 is in direct contact with the leg 110.

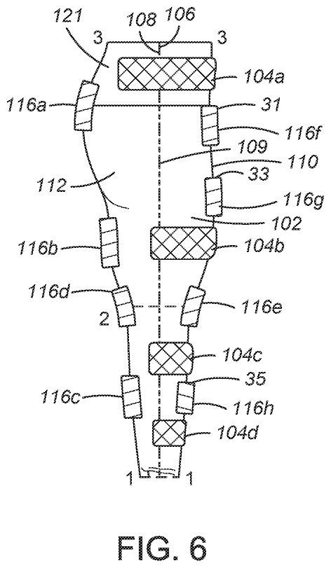

Another exemplary embodiment of the side perspective view of the thermal treatment device maintained on the human leg in accordance with another embodiment of the present disclosure is shown in FIG. 6. In an aspect of the present disclosure, the split sleeve 102 may further comprise a plurality of pockets configured at a location on the top and back of the leg 110. In an exemplary embodiment the split sleeve 102 may comprise a plurality of pockets, a sixth pocket 116f adjacent to an upper thigh, a seventh pocket 116g adjacent to a lower thigh, and an eighth pocket 116h adjacent to a shin of the leg 110 on the outside surface 121 of the sleeve 102 as shown in the FIG. 6. In accordance with the present disclosure, the fourth pocket 116d may be located behind the kneecap and the fifth pocket 116e may be located at the kneecap area for receiving a thermal source and provide heat treatment to the knee. When the sleeve 102 is secured on the leg 110, the inner side 131d (FIG. 3) of the fourth pocket 116d and the inner side 131f (FIG. 3) of the fifth pocket 116e are in direct contact with the leg 110. Particularly, the inner side 131d of the fourth pocket 116d is in direct contact with the underside area of the knee and the inner side 131f of the fifth pocket 116e is in direct contact with the kneecap area of the leg 110.

In an exemplary embodiment, the fifth pocket 116e located at the kneecap area has a smaller size than the first pocket 116a, the second pocket 116b, the third pocket 116c, the fourth pocket 116d, the sixth pocket 116f, the seventh pocket 116g, and the eighth pocket 116h. In an aspect, the fifth pocket 116e located at the kneecap area may have an equal size as the fourth pocket 116d located behind the kneecap. The fourth pocket 116d located behind the kneecap and the fifth pocket 116e located at the kneecap area are located on the knee area to cover the knee from the top and bottom to provide double thermal treatment to the knee. For accessibility, the sixth pocket 116f may be open on the top side 31, the seventh pocket 116g may be open on the top side 33, and the eighth pocket 116h may be open on the top side 35 from the outside surface 121 of the sleeve 102. Alternatively, the sixth pocket 116f, the seventh pocket 116g, and the eighth pocket 116h may be open on any side as per suitability. The rest of the elements are the same as described in FIG. 4 previously.

In another aspect of the present disclosure, the thermal treatment device 101 of the present disclosure may comprise a non-split sleeve 102 with pockets to cover the human leg. The non-split sleeve 102 may comprise a first open end at the waistline 3-3 and a second open end at the ankle line 1-1. The non-split sleeve 102 may be worn like a sleeve from the first open end at the waistline 3-3 and the second open end at the ankle line 1-1 to cover the leg 110.

The thermal treatment device 101 of the present disclosure with the sleeve 102 provides an effective device for treating sciatica. The thermal treatment device 101 may also be integrated into the existing or known products and used in combination with other devices. The sleeve 102 of the thermal treatment device 101 comprises at least three pockets the first pocket 116a adjacent to the glute, the second pocket 116b adjacent to the hamstring, and the third pocket 116c adjacent to the calf of the leg 110 when the sleeve is on the leg. A thermal source can be placed at least in the first pocket 116a, the second pocket 116b, and the third pocket 116c and the pockets are in direct contact with the leg 110. The thermal source provides a thermal effect to the sciatic nerve. The position on the sleeve 102 of the first pocket 116a near to and in contact with the glute, the second pocket 116b near to and in contact with the hamstring, and the third pocket 116c near to and in contact with the calf is judiciously located to effectively provide relief to the sciatic nerve which runs the whole length of the leg. The plurality of bands, the first band 104a, the second band 104b, the third band 104c, and the fourth band 104d and the design of the split sleeve 102 with the belt 103 secures the device 101 at its intended place on the leg 110.

Targeted thermal treatment of a plurality of muscles, ligaments, tendons, and other tissue on the leg and back simultaneously with a thermal medium effectively treats and provides relief to a person suffering from sciatic pain. The sleeve 102 of the thermal treatment device 101 may comprise the fourth pocket 116d on the back of the knee and the fifth pocket 116e on the top of the kneecap for receiving the thermal source. The sleeve 102 may also be used to provide the thermal treatment to the knee. Also, when wrapped around the leg 110 with the first band 104a, the second band 104b, the third band 104c, and the fourth band 104d of the sleeve 102 may provide compression treatment to the leg 110. The sleeve when wrapped around the leg 110, may distribute pressure over some or all of a surface of the leg 110, in all directions, thereby significantly lowering the pressure or weight on a given region.

In accordance with the present disclosure, a method of thermally treating a human leg is also disclosed. The leg 110 is covered with the sleeve 102 as shown in FIG. 1. The sleeve 102 is wrapped around the leg 110 by placing the sleeve 102 on the leg 110. The sleeve 102 is fastened to the leg 110 by wrapping the first band 104a, the second band 104b, the third band 104c, and the fourth band 104d from the first side 114 to the second side 124 and engaging the fasteners on the second end 14 of the first band 104a, the second end 24 of the second band 104b, the second end 34 of the third band 104c, and the second end 44 of the fourth band 104d with the patch or strip 105 of mating fasteners on the second side 124 of the sleeve 102 to secure it on the leg 110. A thermal source is placed in at least the three pockets, the first pocket 116a adjacent to the glute, the second pocket 116b adjacent to the hamstring, and the third pocket 116c adjacent to the calf of the leg 110 as shown in FIG. 4. In accordance with an exemplary embodiment, the thermal source is a gel pack. Gel packs can be heated by microwave or cooled by refrigeration. Suitable gel packs are available from Gelpax.com in Ontario, Canada.

In the method, first, a cooled thermal source is placed in each of the first pocket 116a adjacent to the glute, the second pocket 116b adjacent to the hamstring, and the third pocket 116c adjacent to the calf of the leg 110 to provide a cooling effect to the leg 110 or the sciatic nerve in particular. The thermal source can be cooled in a freezer or refrigerator or may contain ice. Thereafter, the cooled thermal source is placed inside each of the first pocket 116a adjacent to the glute from the open side 11, the second pocket 116b adjacent to the hamstring from the open side 13, and the third pocket 116c adjacent to the calf from the open side 15. The cooled thermal source inside the first pocket 116a is adjacent to the glute, the cooled thermal source inside the second pocket 116b is adjacent to the hamstring, and the cooled thermal source inside the third pocket 116c is adjacent to the calf to provide cooling to the glute, the hamstring, and the calf of the leg 110, respectively. The cooled thermal source is maintained on the leg 110 within the pockets of the split sleeve 102 for a first predetermined period of time such as 5 to 15 minutes. Thereafter, the cooled thermal source is removed from the leg 110 such as by removing the cooled thermal source from each of the open side 11 of the first pocket 116a, from the open side 13 of the second pocket 116b, and from the open side 15 of the third pocket 116c. The cooling of the leg tissue diminishes inflammation. Thereafter, a heated thermal source is contacted with the leg 110. The thermal source may be a pack of material that is heated in a microwave oven or in heated water. A heated thermal source is placed inside each of the first pocket 116a adjacent to the glute through the open side 11, the second pocket 116b adjacent to the hamstring through the open side 13, and the third pocket 116c adjacent to the calf through the open side 15 to provide a heating effect to the leg 110 or the sciatic nerve in particular. Heating the leg tissue causes dilation of the blood vessels, which facilitates blood and oxygen transfer to the affected tissue hastening its relief and repair. The heated thermal source is maintained on the leg 110 within each of the first pocket 116a adjacent to the glute, the second pocket 116b adjacent to the hamstring, and the third pocket 116c adjacent to the calf of the sleeve 102 for a second predetermined period of time such as 5 to 15 minutes. The heated thermal source transfers the heat to the leg 110 or the sciatic nerve in particular. Thereafter, the heated thermal source is removed from the leg 110 such as by removing the heated thermal source from each of the open side 11 of the first pocket 116a, from the open side 13 of the second pocket 116b, and from the open side 15 of the third pocket 116c.

The sleeve 102 can also provide thermal treatment to the knee. A heated or a cooled thermal source can be placed inside the fourth pocket 116d to provide the thermal effect behind the knee or in the fifth pocket 116e to provide the thermal effect to the kneecap area, or in both the fourth pocket 116d and the fifth pocket 116e. The thermal source is heated or cooled and placed inside either or both of the fourth pocket 116d from the open side 17 and inside the fifth pocket 116e from the open side 19. The thermal source is maintained on the leg 110 within either or both of the fourth pocket 116d and the fifth pocket 116e of the split sleeve 102 for a third predetermined period of time such as 5 to 15 minutes to provide a thermal treatment to the knee area.

The split sleeve 102 can also provide thermal treatment to the thigh. A heated or a cooled thermal source can be placed inside the sixth pocket 116f to provide the thermal effect to the upper thigh or in the seventh pocket 116g to provide the thermal effect to the lower thigh, or inside both the sixth pocket 116f and the seventh pocket 116g. The thermal source is heated or cooled and placed inside the sixth pocket 116f from the open side 31 and inside the seventh pocket 116g from the open side 33. The thermal source is maintained on the leg 110 within either or both of the sixth pocket 116f and the seventh pocket 116g of the split sleeve 102 for a fourth predetermined period of time such as 5 to 15 minutes to provide a thermal treatment to the thigh of the leg 110.

Further, the split sleeve 102 can also provide thermal treatment to the shin. A heated or a cooled thermal source can be placed inside the eight pocket 116h to provide the thermal effect to the shin. The thermal source is heated or cooled and placed inside the eighth pocket 116h from the open side 35. The thermal source is maintained on the leg 110 within the eighth pocket 116h of the split sleeve 102 for a fifth predetermined period of time such as 5 to 15 minutes to provide a thermal treatment to the shin of the leg 110.

Provided is a device for effectively administering thermal treatment to the leg to address a sciatic nerve condition and a method of administering thermal treatment to relieve the pain suffered by the sciatic nerve.

### Specific Embodiments

While the following is described in conjunction with specific embodiments, it will be understood that this description is intended to illustrate and not limit the scope of the preceding description and the appended claims.

A first embodiment of the present disclosure is a thermal treatment device, comprising a sleeve for covering a back of a human leg; and the sleeve has a first pocket located adjacent to a glute, a second pocket adjacent to a hamstring, and a third pocket adjacent to a calf of the leg when the sleeve covers the back of the human leg, the pockets adaptable to receive a thermal source. The thermal treatment device of claim 1 wherein the sleeve covers the back of the leg from a calf to the glute. The thermal treatment device of claim 1 wherein the thermal source is selected from one or more of a gel pack. The thermal treatment device of claim 1 further comprising a plurality of bands for wrapping the sleeve around the leg. The thermal treatment device of claim 1 wherein the sleeve is a split sleeve having a first edge and a second edge that are adjacent to each other when the split sleeve is wrapped around the leg. The thermal treatment device of claim 5 wherein the split sleeve, when wrapped around the leg, encases the leg such that the first edge of the split sleeve is opposed to the second edge of the split sleeve. The thermal treatment device of claim 1 wherein the sleeve has an inside surface and an outside surface, and wherein the inside surface is in direct contact with the leg. The thermal treatment device of claim 7 wherein the pockets are located on the outside surface of the sleeve. The thermal treatment device of claim 7 wherein the pockets are accessible on the outside surface of the sleeve. The thermal treatment device of claim 1 further comprising a fourth pocket adjacent to an underside of a knee and a fifth pocket adjacent to a kneecap. The thermal treatment device of claim 1 further comprising a sixth pocket adjacent to an upper thigh, a seventh pocket adjacent to a lower thigh. The thermal treatment device of claim 1 further comprising an eighth pocket adjacent to a shin of the leg. The thermal treatment device of claim 1, wherein the sleeve provides compression to the leg when maintained on the leg. The thermal treatment device of claim 4 wherein the band includes a patch of hook-and-loop fasteners. The thermal treatment device of claim 4 further comprising a band that extends across a human waist.

A second embodiment of the present disclosure is a method of thermally treating a human leg, comprising placing a first cooled thermal source adjacent to a glute, placing a second cooled thermal source adjacent to a hamstring, and placing a third cooled thermal source adjacent to a calf of a human leg; removing the first cooled thermal source from adjacent to the glute, removing the second cooled thermal source from adjacent to the hamstring, and removing the third cooled thermal source from adjacent to the calf of the human leg; and placing a first heated thermal source adjacent to the glute, placing a second heated thermal source adjacent to the hamstring, and placing a third heated thermal source adjacent to the calf of the human leg. An embodiment of the present disclosure is one, any or all of prior embodiments in this paragraph up through the second embodiment in this paragraph further comprising covering a back of a human leg with a sleeve comprising at least three pockets, a first pocket located adjacent to a glute, a second pocket adjacent to a hamstring, and a third pocket adjacent to a calf of the leg, wherein the thermal source is placed inside the first pocket, the second pocket, and the third pocket.

A third embodiment of the present disclosure is a thermal treatment device, comprising a sleeve for covering a back of a human leg, the sleeve has a first pocket located adjacent to a glute, a second pocket adjacent to a hamstring, and a third pocket adjacent to a calf of the leg when the sleeve covers the back of the human leg, the pockets adaptable to receive a thermal source; and a plurality of bands for wrapping the sleeve around the leg. An embodiment of the present disclosure is one, any or all of prior embodiments in this paragraph up through the third embodiment in this paragraph wherein the sleeve is a split sleeve having a first edge and a second edge, the split sleeve is configured to be wrapped around the leg to cover the back of the leg from the calf to the glute. The thermal treatment device of claim 18 wherein the split sleeve, when wrapped around the leg, encases the leg such that the first edge of the split sleeve is opposed to the second edge of the split sleeve.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the present disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

The invention claimed is:

1. A thermal treatment device, comprising:
a sleeve for covering a back of a human leg; and
the sleeve has a first pocket located adjacent to a glute, a second pocket adjacent to a hamstring, and a third pocket adjacent to a calf of the leg when the sleeve covers the back of the human leg, said pockets adaptable to receive a thermal source.

2. The thermal treatment device of claim 1 wherein the sleeve covers the back of the leg from a calf to the glute.

3. The thermal treatment device of claim 1 wherein the thermal source can be a gel pack.

4. The thermal treatment device of claim 1 further comprising a plurality of bands for wrapping the sleeve around the leg.

5. The thermal treatment device of claim 1 wherein the sleeve is a split sleeve having a first edge and a second edge that are adjacent to each other when the split sleeve is wrapped around the leg.

6. The thermal treatment device of claim 5 wherein the split sleeve, when wrapped around the leg, encases the leg such that the first edge of the split sleeve is opposed to the second edge of the split sleeve.

7. The thermal treatment device of claim 1 wherein the sleeve has an inside surface and an outside surface, and wherein the inside surface is in direct contact with the leg.

8. The thermal treatment device of claim 7 wherein the pockets are located on the outside surface of the sleeve.

9. The thermal treatment device of claim 7 wherein the pockets are accessible on the outside surface of the sleeve.

10. The thermal treatment device of claim 1 further comprising a fourth pocket adjacent to an underside of a knee and a fifth pocket adjacent to a kneecap.

11. The thermal treatment device of claim 1 further comprising a sixth pocket adjacent to an upper thigh, a seventh pocket adjacent to a lower thigh.

12. The thermal treatment device of claim 1 further comprising an eighth pocket adjacent to a shin of the leg.

13. The thermal treatment device of claim 1, wherein the sleeve provides compression to the leg when maintained on the leg.

14. The thermal treatment device of claim 4 wherein the bands include a patch of hook-and-loop fasteners.

15. The thermal treatment device of claim 4 further comprising a band that extends across a human waist.

16. A method of thermally treating a human leg, comprising:
placing a first cooled thermal source adjacent to a glute, placing a second cooled thermal source adjacent to a hamstring, and placing a third cooled thermal source adjacent to a calf of a human leg;
removing the first cooled thermal source from adjacent to the glute, removing the second cooled thermal source from adjacent to the hamstring, and removing the third cooled thermal source from adjacent to the calf of the human leg; and
placing a first heated thermal source adjacent to the glute, placing a second heated thermal source adjacent to the hamstring, and placing a third heated thermal source adjacent to the calf of the human leg.

17. The method of claim 16 further comprising:
covering a back of a human leg with a sleeve comprising at least three pockets, a first pocket located adjacent to a glute, a second pocket adjacent to a hamstring, and a third pocket adjacent to a calf of the leg,
wherein the thermal sources are placed inside the first pocket, the second pocket, and the third pocket.

18. A thermal treatment device, comprising:
a sleeve for covering a back of a human leg, the sleeve has a first pocket located adjacent to a glute, a second pocket adjacent to a hamstring, and a third pocket adjacent to a calf of the leg when the sleeve covers the back of the human leg, said pockets adaptable to receive a thermal source; and
a plurality of bands for wrapping the sleeve around the leg.

19. The thermal treatment device of claim 18 wherein the sleeve is a split sleeve having a first edge and a second edge, the split sleeve is configured to be wrapped around the leg to cover the back of the leg from the calf to the glute.

20. The thermal treatment device of claim 18 wherein the split sleeve, when wrapped around the leg, encases the leg such that the first edge of the split sleeve is opposed to the second edge of the split sleeve.
